(19) United States
(12) Patent Application Publication
(10) Pub. No.: US 2019/0159662 A1
(43) Pub. Date: May 30, 2019

(54) Ultrathin-Caliber Endoscopic Instrument

(71) Applicants: Ralph Said Papas, Beirut (LB); Zygmunt Marek Niewiadomski, Rabieh (Metn) (LB)

(72) Inventors: Ralph Said Papas, Beirut (LB); Zygmunt Marek Niewiadomski, Rabieh (Metn) (LB)

(21) Appl. No.: 16/127,060

(22) Filed: Sep. 10, 2018

Related U.S. Application Data
(60) Provisional application No. 62/592,372, filed on Nov. 29, 2017.

(51) Int. Cl. A61B 1/00; A61B 1/05; A61B 1/07; A61B 1/06

(52) U.S. Cl. CPC A61B 1/00167; A61B 1/00096; A61B 1/00087; A61B 1/0057; A61B 1/07; A61B 1/0676; A61B 1/05

(57) ABSTRACT

An ultrathin-caliber endoscopic instrument, which can be used in various clinical scenarios, such as in embryo transfer procedures. The endoscopic instrument comprises a tubular body and imaging elements for optical imaging or ultrasound imaging. The tubular body may be ultra-thin caliber, for example, having an outer diameter of about 1 mm. There is a working channel extending through the tubular body. An embryo transfer catheter may be passed through the working channel. Images are transmitted via a wireless radio transmitter, such as a Wi-Fi transmitter. The endoscopic instrument may be designed to be fully or semi-disposable after use.

### RELATED APPLICATIONS

This application claims the benefit of U.S. Provisional Patent Application No. 62/592,372 filed on 29 Nov. 2017, the contents of which are incorporated herein by reference.

### TECHNICAL FIELD

This invention relates to endoscope instruments.

### BACKGROUND

In assisted reproduction, the transfer of the embryo into the uterine cavity is the final critical step in a long sequence of events that make up the process cycle. Embryo transfer is performed by loading the embryo into a long, thin catheter. The embryo transfer catheter is then inserted into the outer cervical os, through the cervical canal, out through the inner cervical os, and into the uterine cavity. Here, the embryos are expelled out of the catheter so that they can implant in the endometrial lining. Often, this embryo transfer is performed blindly, guided only by “clinical touch” without direct visualization of the anatomy. In other cases, abdominal ultrasound may be used to help guide the procedure.

Because the cervical canal and inner cervical os are the narrowest parts of the cervical tract, passing the catheter through these areas is the most challenging part of the procedure. A misstep here could injure the cervical tract, which would result in a significantly decreased chances of pregnancy.

### SUMMARY

This invention provides an endoscopic instrument (referred to herein as a “catheterscope”), which could be used in various clinical scenarios that would benefit from ultrathin-caliber endoscopy. One particular clinical scenario where the endoscopic instrument may be particularly useful is an embryo transfer procedure in assisted reproduction. In one aspect, the invention is an endoscopic instrument comprising a tubular body. At the distal end of the tubular body, there is a white light illuminator for providing illumination light for the area being viewed. Extending through the tubular body is an optical fiber bundle for optical imaging.

The tubular body extends out from a housing. Inside the housing is an imaging assembly, which comprises a camera module that receives optical images transmitted through the optical fiber bundle. The imaging assembly further comprises a short range radio transmitter that is coupled to the camera module and wirelessly transmits image data.

In another aspect, the invention is an endoscopic instrument comprising a tubular body with an ultrasound transducer at its distal end. The tubular body extends out from a housing. Inside the housing is an imaging assembly, which comprises a digital signal processor that receives ultrasound image signals from the ultrasound transducer. The imaging assembly further comprises a short range radio transmitter that is coupled to the digital signal processor and wirelessly transmits ultrasound image data.

General Features:

The tubular body of the endoscopic instrument may be ultrathin-caliber. In some embodiments, the tubular body has an outer diameter of less than 2.5 mm; in some cases, less than 2.0 mm; in some cases, less than 1.5 mm; in some cases, about 1 mm; and in some cases, less than 1.0 mm. The tubular body has an outer diameter of at least 0.1 mm. The tubular body has a working channel to allow for catheter instruments to be passed through. In some embodiments, the working channel has an internal diameter of less 1.5 mm; and in some cases, less than 1.0 mm. The working channel has an internal diameter of at least 0.1 mm. In some embodiments, the tubular body does not have a separate channel for suction, irrigation, or insufflation. In some embodiments, the working channel of the tubular body is the only hollow channel in the tubular body.

The endoscopic instrument may be designed for particular use in an embryo transfer procedure in assisted reproduction. As such, in some embodiments, the tubular body has a length in the range of 12-35 cm; and in some cases, 15-28 cm. The optical fiber bundle in the endoscopic instrument may be made of any suitable material including silica glass or polymer (such as poly(methyl methacrylate) (PMMA) or polystyrene). In some embodiments, the optical fiber bundle is a polymer optical fiber bundle.

As used herein, “short range radio transmitter” means a radio transmitter that transmits radio signals having a range of less than 250 meters, such as those built according to the Wi-Fi or Bluetooth standard. The radio transmitter may communicate with any suitable external, wireless-capable display system for viewing the images, including specialized monitor display systems, or some commonly available electronic devices (e.g. smartphone, tablet computer, notebook computer, etc.). In some embodiments, raw image data from the electronic image sensor of the camera module is fed directly into the radio transmitter for wireless transmission out to the external display device. Raw image data contains the individual readout from each of the electronic image sensor's pixels, with minimal or no processing. By offloading further image processing to the external display device that receives the image data, power demand can be reduced.

The imaging module further comprises a battery, which may be rechargeable or otherwise. The endoscopic instrument may rely solely on battery power for operation. In some embodiments, the endoscopic instrument lacks a connector (e.g. outlet, plug, port, socket, etc.) for an external power supply. The imaging module could also be equipped with an accelerometer (e.g. to detect linear acceleration or 3-axis acceleration). This feature may be useful for establishing an artificial horizon to keep the image correctly oriented relative to the ground. This feature could be turned on/off by the user.

The tubular body may be flexible. In some embodiments, the flexibility range of the tubular body, having a length L, is limited to a droop of less than 0.5×L from the straight horizontal axis; in some cases, less than 0.4×L; in some cases, less than 0.3×L; and in some cases, less than 0.2×L. In some embodiments, the flexibility range of the tubular body is limited to a droop of less than 10 cm from the straight horizontal axis; and in some cases, less than 7 cm. In some embodiments of the invention, the distal end of the tubular body is not steerable.

In another aspect, the invention is an endoscopic instrument comprising a tubular body that is steerable. At the distal end of the tubular body, there are one or more microfluidic actuators that operate to bend the distal end of the tubular body. Each microfluidic actuator is connected to a hydraulic line. In some embodiments, the endoscopic instrument further comprises a syringe port for each of the microfluidic actuators, and connected to the hydraulic line for its microfluidic actuator. In embodiments having two or more such microfluidic actuators, the actuators may be spaced apart evenly around the central axis of the tubular body (for example, two that are spaced 180° apart axially, or three that are spaced 120° apart axially).

In some embodiments, the endoscopic instrument is provided as part of an embryo transfer procedure kit that further comprises an embryo transfer catheter. The working channel of the tubular body is sized to fit the embryo transfer catheter (to allow the catheter to be passed through the working channel).

In another aspect, the invention is a method of performing an embryo transfer procedure in assisted reproduction using an endoscopic instrument described herein. An embryo is loaded onto an embryo transfer catheter. The tubular body of the endoscopic instrument is inserted into the patient's vagina and advanced into the external cervical os. While viewing video images from the endoscopic instrument within the cervical canal, the tubular body is advanced through the cervical canal, then exiting the cervical canal into the uterine cavity. The embryo transfer catheter is inserted into the working channel of the endoscopic instrument, and advanced through the working channel so that it enters into the uterine cavity. Inside the uterine cavity, the embryo is released from the transfer catheter.

In some embodiments, the tubular body is advanced through the external cervical os without prior dilation of the cervix. In some embodiments, after use, the tubular body is detached and disposed. In some embodiments, the entire instrument is disposed after use.

### BRIEF DESCRIPTION OF THE DRAWINGS

FIGS. 1A-C show an example of a catheterscope instrument according to the invention. FIG. 1A shows a wide perspective view; FIG. 1B shows a close-up perspective view of the proximal end; FIG. 1C shows a close-up perspective view of the distal end.

FIG. 2 shows an end-on view of the distal end of the tubular body of the catheterscope instrument of FIG. 1A.

FIGS. 3A and 3B show cross-section side views of the distal end of the tubular body of the catheterscope instrument of FIG. 1A.

FIG. 4 shows a schematic diagram of the proximal end of the catheterscope instrument of FIG. 1A.

FIGS. 5, 6A, 6B, and 7 show another example of a catheterscope instrument according to the invention.

FIG. 5 shows an end-on view of the distal end of the tubular body of the catheterscope instrument.

FIGS. 6A and 6B show cross-section side views of the distal end of the tubular body of the catheterscope instrument.

FIG. 7 shows a schematic diagram of the proximal end of the catheterscope instrument.

FIGS. 8-10 show another example of a catheterscope instrument according to the invention.

FIG. 8 shows an end-on view of the distal end of the tubular body of the catheterscope instrument.

FIG. 9 shows a cross-section side view of the distal end of the tubular body of the catheterscope instrument.

FIG. 10 shows a schematic diagram of the proximal end of the catheterscope instrument.

FIGS. 11, 12A, and 12B show another example of a catheterscope instrument according to the invention.

FIG. 11 shows an end-on view of the distal end of the tubular body of the catheterscope instrument.

FIGS. 12A and 12B show cross-section side views of the tubular body of the catheterscope instrument.

FIGS. 13A-C illustrate an example of how the tubular body may be flexible.

FIGS. 14A and 14B show an example of how a catheterscope of the invention could be used for an embryo transfer procedure.

### DETAILED DESCRIPTION

To assist in understanding the invention, reference is made to the accompanying drawings to shown by way of illustration specific embodiments in which the invention may be practiced.

### 1. Optical Video Imaging with LED Illumination

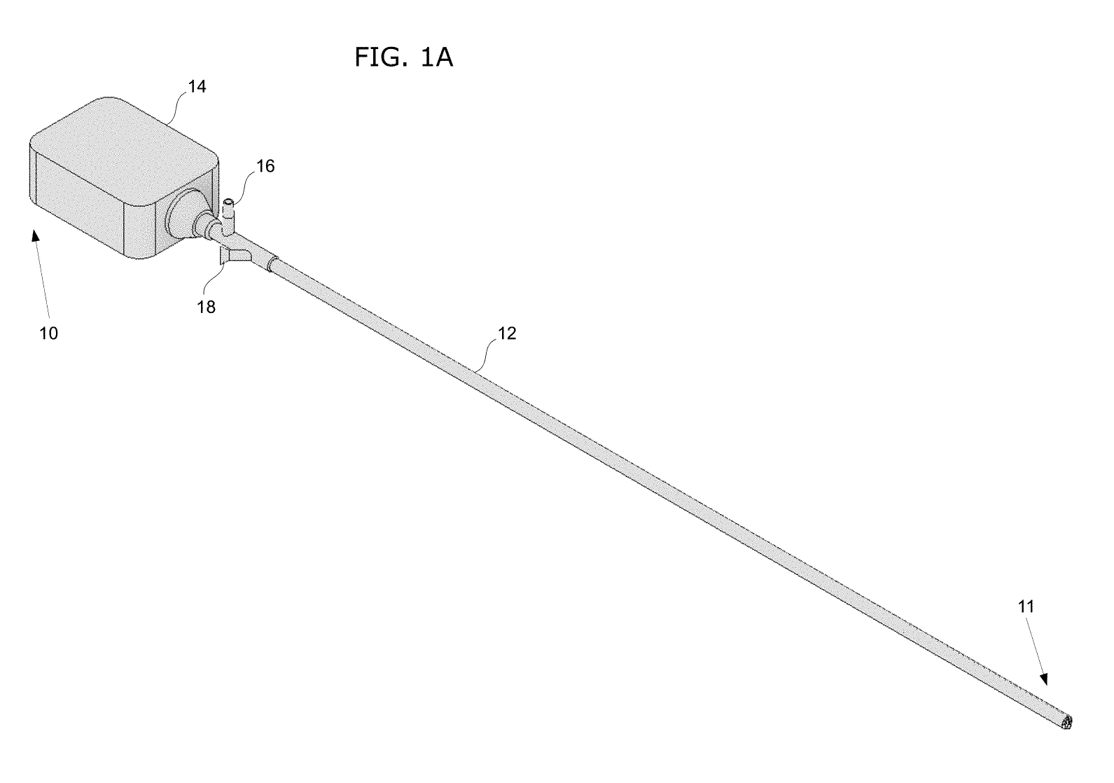

FIG. 1A-C show an example of a catheterscope instrument according to the invention. FIG. 1A shows a wide perspective view of the catheterscope instrument, which comprise an elongate tubular body 12 with a distal end 11. At its proximal end 10, the instrument comprises a housing 14 that is coupled to the tubular body 12. FIG. 1B shows a close-up perspective view of the proximal end 10 of the catheterscope instrument. As seen here, there is a connector port 16 to the suction/irrigation channel 50 of the tubular body 12. There is also an access port 18 to the working channel 40 of the tubular body 12.

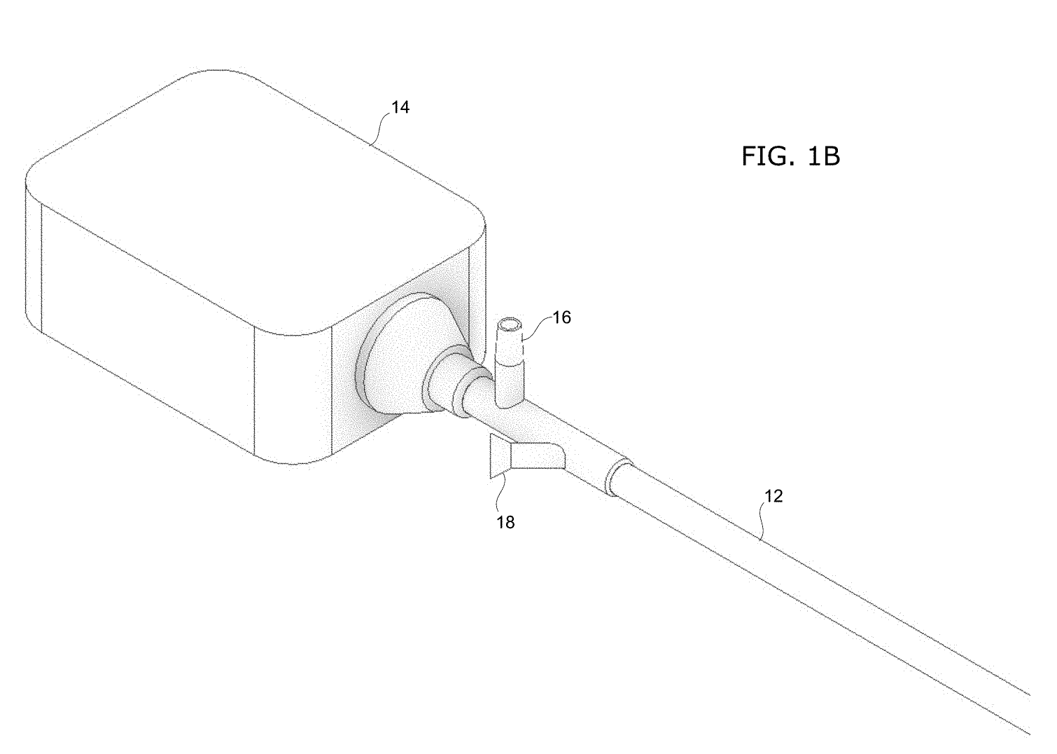

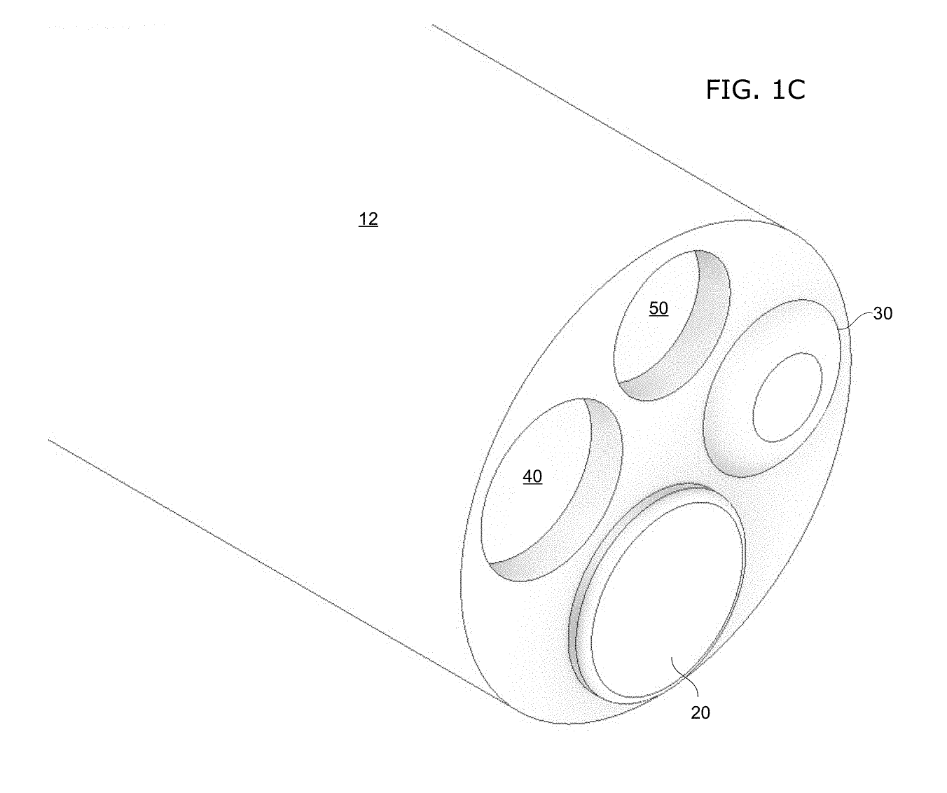

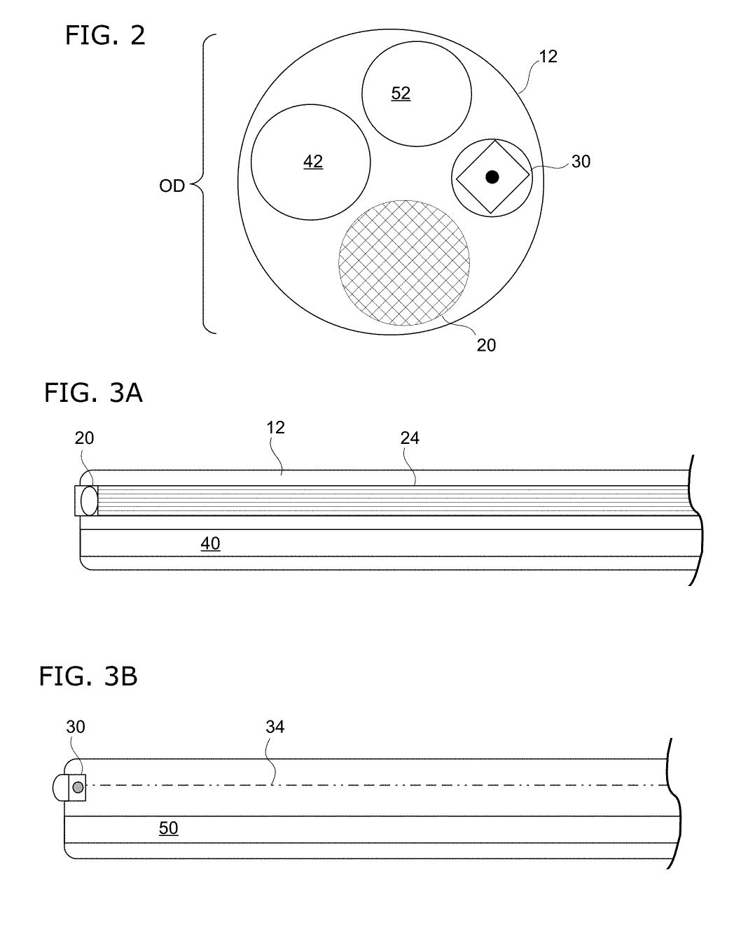

FIG. 1C shows a close-up perspective view of the distal end 11 of the catheterscope instrument. As seen here, there is a working channel 40, a channel for suction/irrigation 50, an LED (light-emitting diode) element 30 for illumination, and lens 20 for the optical fiber bundle 24. Various catheter-type instruments could be inserted through the working channel 40, such as an embryo transfer catheter. FIG. 2 shows an end-on view of the distal end of the tubular body 12. As seen here, the tubular body 12 has an outer diameter (OD) of about 1 mm. There is a distal opening 42 of the working channel 40 and a distal opening 52 of the suction/irrigation channel 50. The LED element 30 provides illumination of the area being imaged. For imaging, there is a lens 20 mounted to the distal end of the optical fiber bundle 24.

FIGS. 3A and 3B show cross-section side views of the distal end of the tubular body 12. As seen in FIG. 3A, the optical fiber bundle 24 and the working channel 40 extends through the length of the tubular body 12. Optical images are captured at the lens 20 and transmitted through the optical fiber bundle 24. FIG. 3B provides a different rotational view to show the LED element 30, its power supply line 34, and the suction/irrigation channel 50, which extends through the length of the tubular body 12.

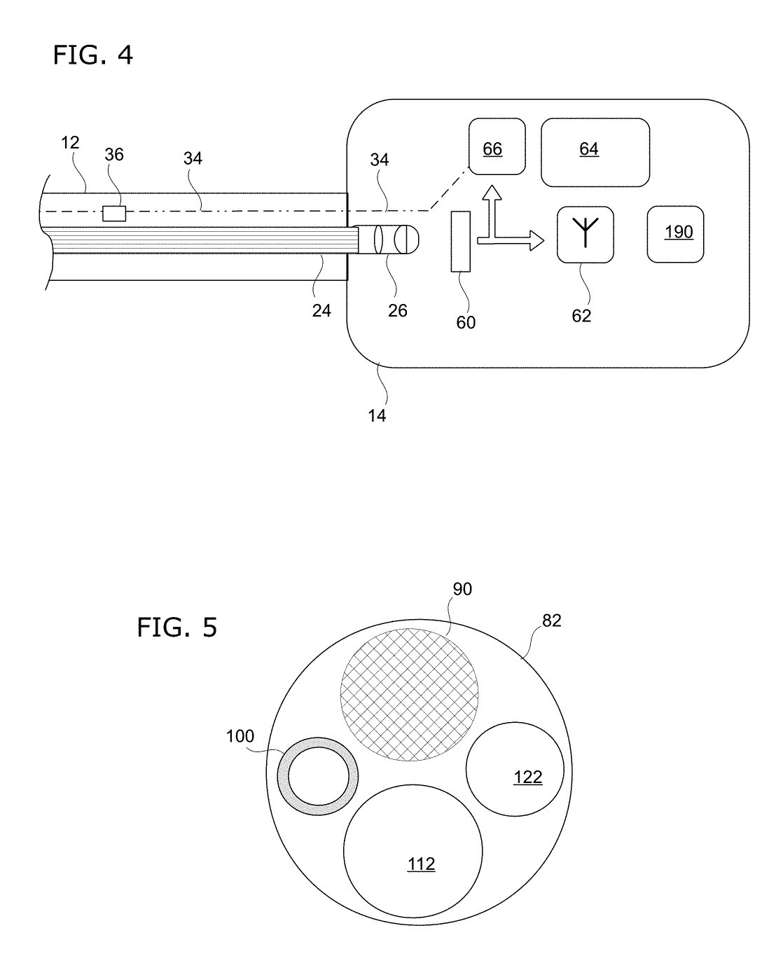

FIG. 4 shows a schematic diagram of the proximal end of the catheterscope instrument. Shown here is the housing 14 which houses the imaging module with its electronic and electro-optical components. At the proximal end of the optical fiber bundle 24, a lens assembly 26 is seated thereon. Via the lens assembly 26, the images are projected onto an electronic image sensor 60 of the camera module. The camera module has autofocus capability and electronic image sensor 60 is selected for maximizing sensitivity and resolution at the smallest image sensor size. The autofocus system can give the ability to compensate for intrinsic defects in the miniaturized optical components.

Image data from sensor 60 is transmitted out wirelessly via a Wi-Fi transmitter 62 so that the images can be received by the clinician using a wireless-capable display device, such as a tablet computer or smartphone. Meanwhile, the image data is also fed to an automatic gain control (AGC) circuit 66 to control the brightness of LED 30 illumination. This circuit 66 provides real-time feedback control for illumination brightness to optimize efficient illumination, maintain good image visibility, and have thermal management.

There is a positive temperature coefficient (PTC) thermistor (not shown) to prevent overcurrent conditions that could result in overheating. Additionally, interrupting the power supply line 34 is an appropriately-selected fuse 36 to prevent any overcurrent. Housing 14 also contains a battery 64 that serves as a power supply for the components. The imaging module is also equipped with a gravity sensor 190 (accelerometer) that can be used to establish an artificial horizon. Output from the gravity sensor 190 could be used to process images such that they are displayed upright relative to the ground.

### 2. Optical Video Imaging with Phosphor Illumination

FIGS. 5, 6A, 6B, and 7 show another example of a catheterscope instrument according to the invention. FIG. 5 shows an end-on view of the distal end of the tubular body 82. The tubular body 82 has an outer diameter of about 1 mm. There is a distal opening 112 of the working channel 110. A phosphorescent element 100 provides illumination of the area being imaged. For imaging, there is a lens 90 mounted to the distal end of the optical fiber bundle 94.

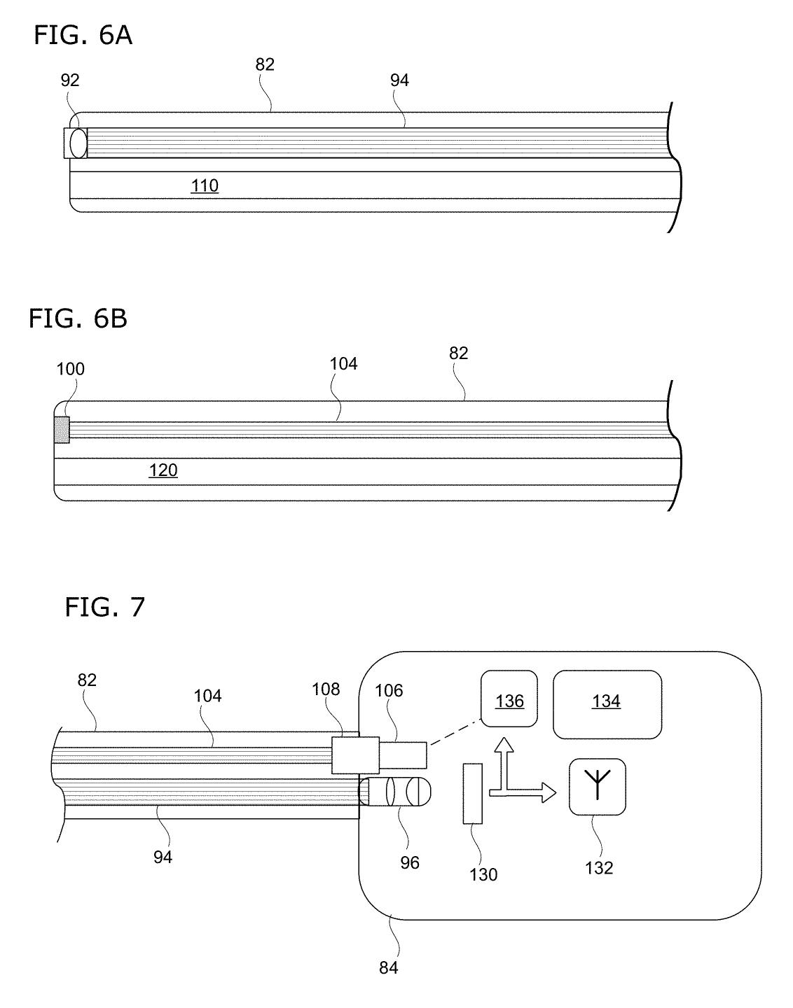

FIGS. 6A and 6B show cross-section side views of the distal end of the tubular body 82. As seen in FIG. 6A, the optical fiber bundle 94 and the working channel 110 extends through the length of the tubular body 82. Optical images are captured at the lens 90 and transmitted through the optical fiber bundle 94. FIG. 6B provides a different rotational view to show the separate optical fiber 104 (single fiber) that is used for transmitting green laser light (or other suitable wavelength) that excites the phosphorescent element 100 to produce illumination light. There is also a suction/irrigation channel 120, which extends through the length of the tubular body 82.

FIG. 7 shows a schematic diagram of the proximal end of the catheterscope instrument. Shown here is the housing 84 which houses the imaging module with its electronic and electro-optical components. At the proximal end of the optical fiber bundle 94, a lens assembly 96 is seated thereon. Via the lens assembly 96, the images are projected onto an electronic image sensor 130 of the camera module. The camera module has autofocus capability and electronic image sensor 130 is selected for maximizing sensitivity and resolution at the smallest image sensor size. The autofocus system can give the ability to compensate for intrinsic defects in the miniaturized optical components. Housing 84 also contains a green laser emitter 106 (or other suitable wavelength) that is mounted onto the proximal end of the optical fiber 104 that is fed through a fiber socket 108. Housing 84 also contains a battery 134 that serves as a power supply for the components.

Image data from sensor 130 is transmitted out wirelessly via a Wi-Fi transmitter 132 so that the images can be received by the clinician using a wireless-capable display device, such as a tablet computer or smartphone. Meanwhile, the image data is also fed to an automatic gain control (AGC) circuit 136 to control the brightness of laser emitter 106. This circuit 136 provides real-time feedback control of the green laser 106, which in turn controls the illumination brightness produced by the phosphorescent element 100.

### 3. Ultrasound Imaging

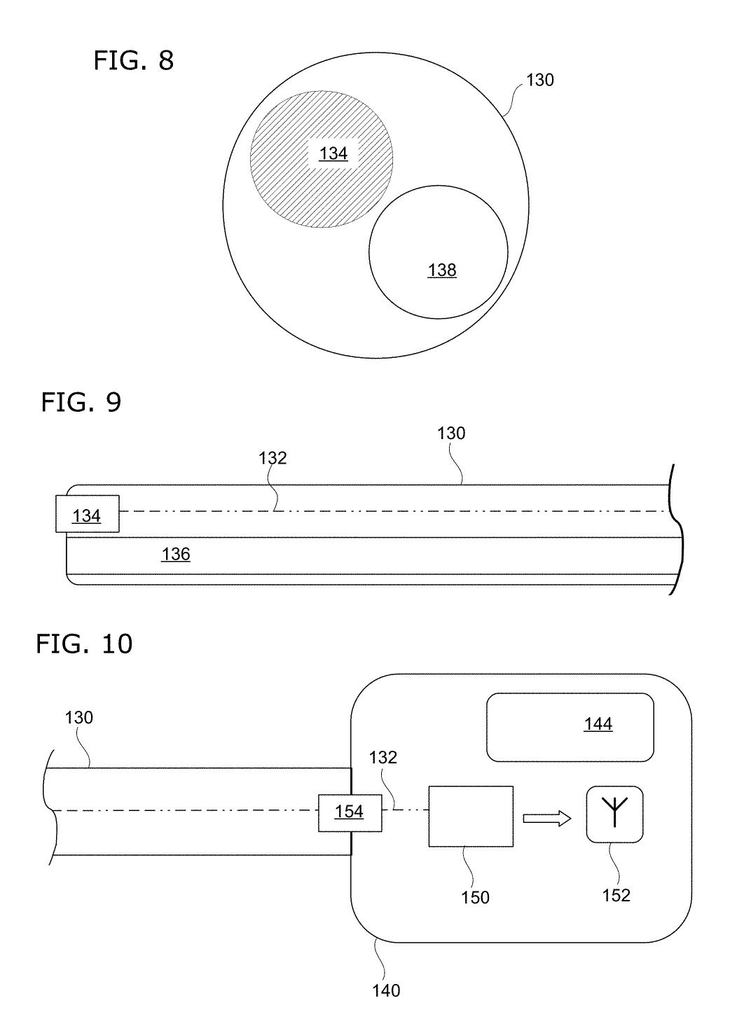

FIGS. 8-10 show another example of a catheterscope instrument according to the invention. FIG. 8 shows an end-on view of the distal end of the tubular body 130. The tubular body 130 has an outer diameter of about 1 mm. There is a distal opening 138 of the working channel 136. For imaging, there is a multispectrum phased array ultrasound imaging transducer 134 mounted on the distal tip of the tubular body 130. FIG. 9 shows a cross-section side view of the distal end of the tubular body 130. Signals from ultrasound transducer 134 are transmitted via signal cable 132. The working channel 136 extends through the length of the tubular body 130.

FIG. 10 shows a schematic diagram of the proximal end of the catheterscope instrument. Shown here is the housing 140 which houses the ultrasound imaging module. The signal cable 132 is fed into the housing 140 via a transducer socket seating assembly 154. The signals from the ultrasound transducer 134 are received by the ultrasound imaging controller that comprises a digital signal processor 150. The processed image data is transmitted out wirelessly via a Wi-Fi transmitter 152 so that the images can be received by the clinician using a wireless-capable display device, such as a tablet computer or smartphone. Housing 140 also contains a battery 144 that serves as a power supply for the components.

### 4. Steerable Tip

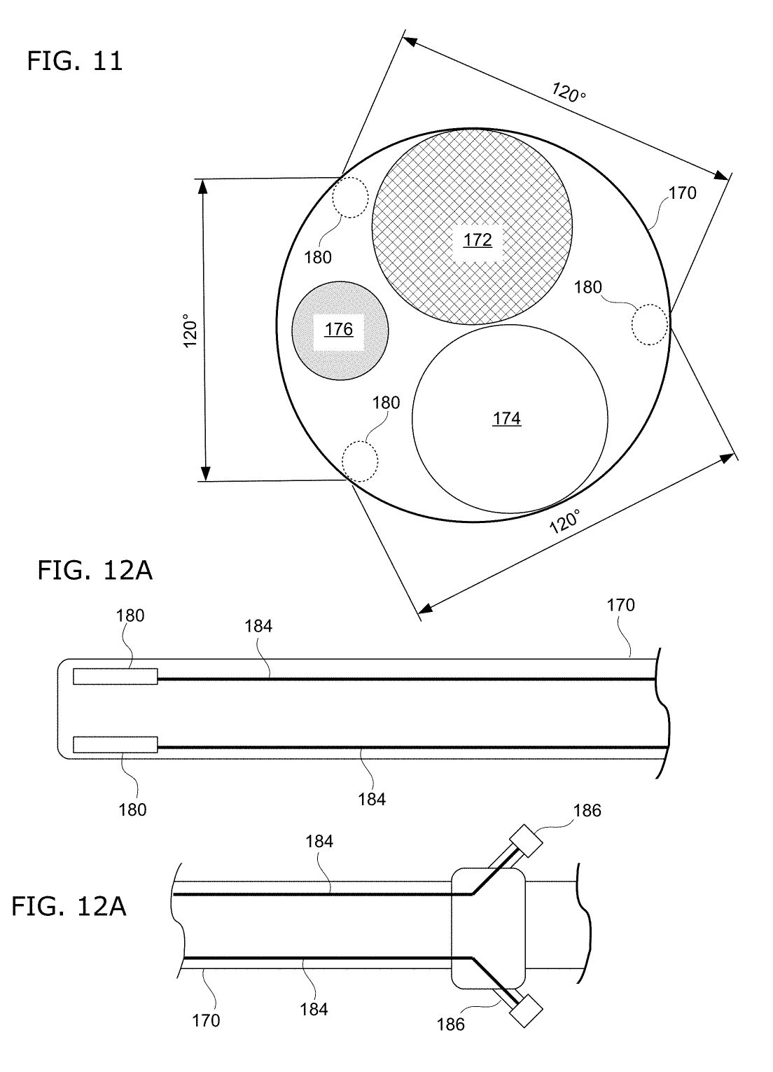

FIGS. 11, 12A, and 12B show another example of a catheterscope instrument according to the invention. FIG. 11 shows an end-on view of the distal end of the tubular body 170. There is a distal opening 174 of the working channel. A white light illumination element 176 provides illumination of the area being imaged. For imaging, there is a lens 172 mounted to the distal end of the optical fiber bundle. Seen in this view are three microfluidic bending actuators 180 that are axially spaced apart at 120°. Activation of one or more of the microfluidic actuators 180 work to bend the distal end of the tubular body 170. The microfluidic actuator 180 may operate in a pull-type or push-type bending. In a pull-type bending, activation of an individual microfluidic actuator 180 causes bending of the distal end towards that particular actuator. In push-type bending, activation of an individual microfluidic actuator 180 causes bending of the distal end away from that particular actuator.

FIGS. 12A and 12B show cross-section side views of the tubular body 170. FIG. 12A shows the distal end of the tubular body 170 (two of the three microfluidic actuators 180 being visible in this view). The microfluidic actuators 180 are each individually connected to its own hydraulic line 184 containing a non-compressible hydraulic fluid. The hydraulic fluid is sterile and biocompatible so that it would be harmless in case of leakage during use. In this particular example, the actuators 180 are operated by pressurizing the hydraulic fluid to bend the distal end and depressurizing to straighten the distal end.

FIG. 12B shows a more proximal portion of the tubular body 170 to illustrate how the microfluidic actuators 180 are triggered. In this particular example, the microfluidic actuators 180 are operated manually using syringes. Each hydraulic line 184 is connected to its own syringe port 186 (two of the three shown in this view). A syringe could be fitted into one or more of the syringe ports 186. The syringe is operated (e.g. by depressing and releasing the plunger) to pressurize/depressurize the hydraulic line 184, thereby triggering the microfluidic actuators 180 to steer the distal end of the tubular body 170. In some embodiments, the volume of hydraulic fluid used for each microfluidic actuators 180 (and its hydraulic line 184) is less than 7 ml. To prevent over-pressure, this system may further comprise pressure limiting mechanism(s), such as load spring(s), pressure relief valve(s), etc. In some embodiments of the invention, a steerable catheterscope instrument is provided as part of an embryo transfer procedure kit that further comprises an embryo transfer catheter and one or more syringes that are prefilled with a sterile, biocompatible hydraulic fluid.

In an alternate embodiment, a pump system is used to pressurize the hydraulic line 184. For example, a manual hand/finger pump could be used. The manual pump system could include a pressure relief valve or flow control valve. In an alternate embodiment, the pump system is mechanized. Such a mechanized pump system may include electric motorized pump(s), pressurized gas cartridge(s), valve(s), or valve controller(s). Such a mechanized pump system could be operated by an activation trigger (e.g. button operation) or remotely by electronic control from the display device via a radio transmitter/receiver. For example, remote electronic control from the display device could allow the user to wirelessly control steering of the catheterscope. In some cases, the mechanized pump system is battery-powered. In some cases, the mechanized pump system is powered by a pressurized regulated gas cartridge.

Microfluidic actuators used in the catheterscope of the invention may be designed for high durability, such as being able to perform reliably for many full bend cycles. In some embodiments, to improve durability, the microfluidic actuators have a length to width ratio of at least 15:1 (length:width); and in some cases, in the range of 15:1 to 50:1.

### 5. Auto-Activation & Power Saving Features

The catheterscope's imaging system may have auto-activation feature(s) or power saving features. In some embodiments, power to the illumination system (e.g. LED element, laser for phosphor element, etc.) can be activated by attachment of the tubular body to the housing and its imaging module (e.g. by contact rings on the tubular body that close the circuit for the illumination power supply). The system may remain active for a specified time before going to power-saving mode, such as shutdown or sleep mode. The power-saving mode may be triggered if the radio transmitter does not have an active connection or if the imaging module is not producing an image. It can be reactivated if needed by removing and reinserting the tubular body section of the catheterscope or by pressing a power button. The system may remain active as long as there is sufficient charge if there is an active WIFI connection. The imaging system may shut down within a short period of time (e.g. one minute) if the tubular body is detached from the socket to the housing for the imaging module.

The imaging module could also be equipped with an accelerometer (e.g. to detect linear acceleration or 3-axis acceleration). This feature may be useful for establishing an artificial horizon to keep the image correctly oriented relative to the ground. This feature could be turned on/off by the user. In some embodiments, raw output from the accelerometer is fed directly into the radio transmitter for wireless transmission out to the external display device. By doing this, the image processing needed to correctly orient the displayed image could be offloaded to the external display device, thereby reducing power demand.

### 6. Flexibility

The tubular body may be flexible so that it can negotiate through the external os and cervical canal. However, the tubular body should also be sufficiently rigid that it can be precisely manipulated from the proximal end of the instrument. In some embodiments, the flexibility range of the tubular body, having a length L, is limited to a droop of less than 0.5×L from the straight horizontal axis at the distal end tip of the tubular body.

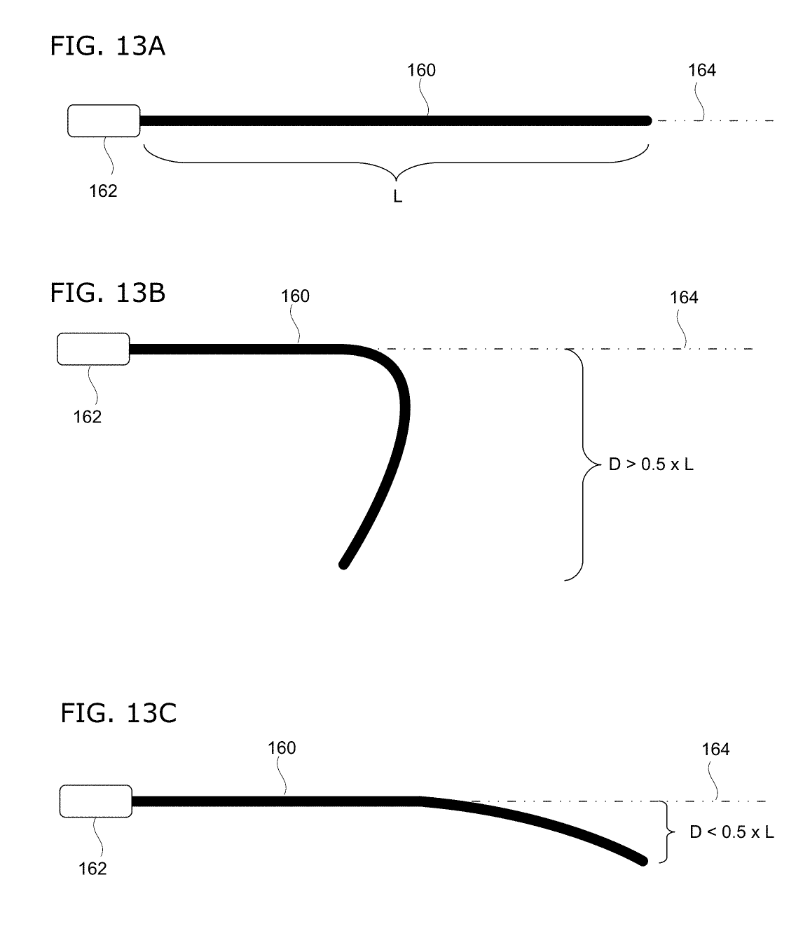

As used herein, the term “droop” means the distance that the distal tip of the tubular body deviates from the straight horizontal axis defined by the immediate direction in which the tubular body extends out from the housing. This measurement is illustrated in FIGS. 13A-C. FIG. 13A shows a rigid (non-flexible) tubular body 160 extending out from the housing 162 along a straight horizontal axis 164. In contrast, FIG. 13B shows a tubular body 160 having too much flexibility, causing significant droop (designated as “D” in the figure) that is greater than 0.5×L. This degree of droop would be typical for conventional gastrointestinal or nasopharyngeal endoscopes. In contrast to both of the preceding, FIG. 13C shows a flexible tubular body 160 of length L having a droop amount of less than 0.5×L from the straight horizontal axis.

In some embodiments of the invention, the flexibility range of the tubular body is limited to a droop of less than 0.4×L from the straight horizontal axis; and in some cases, less than 0.3×L; and in some cases, less than 0.2×L. In some embodiments of the invention, the distal end of the tubular body is not steerable. Again, steerable tips are often features of other conventional, flexible endoscopes.

### 7. Disposable Design

The catheterscope may be designed for fully- or semi-disposable use. In some embodiments, the entire catheterscope is designed to be disposable. In some embodiments, the tubular body is detachable from the housing for the imaging module and the tubular body is disposable (but not the housing and its imaging module). As such, in some embodiments, in a medical procedure that uses the catheterscope, the entire catheterscope may be disposed after use. In some embodiments, after use, the tubular body is detached and disposed (but not the housing and its imaging module).

### 8. Example Use

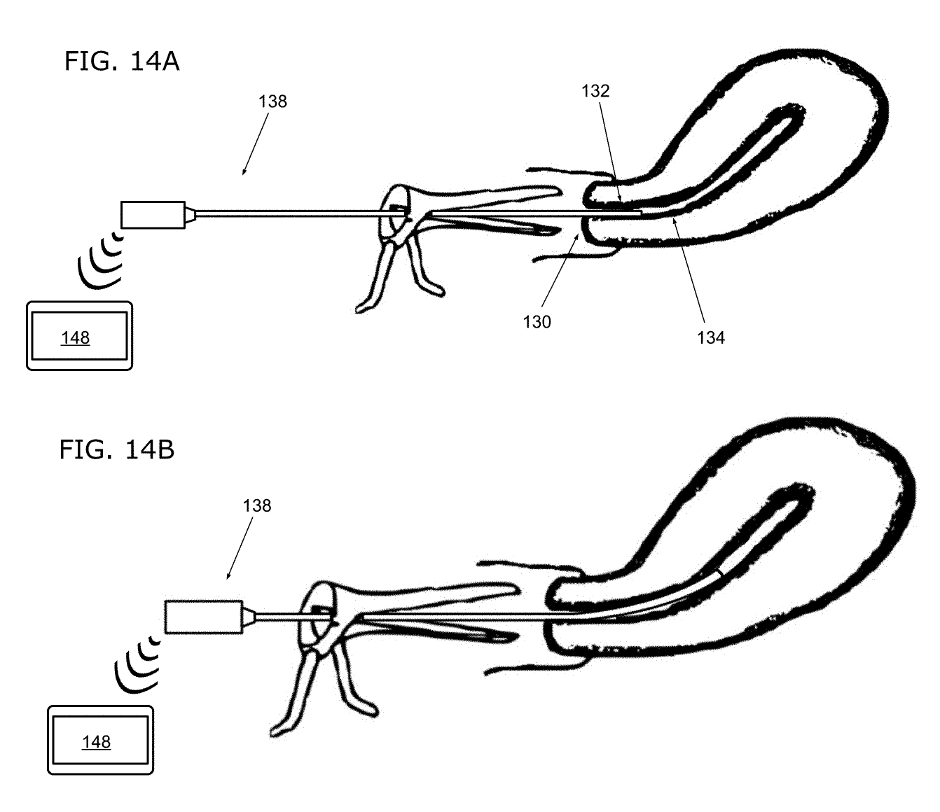

FIGS. 14A and 14B show an example of how a catheterscope of the invention could be used. As shown in FIG. 14A, a speculum is inserted into the vagina to allow examination of the cervix. The distal end of the catheterscope 138 is inserted through the vagina and into the external os 130 of the cervix. The catheterscope 138 transmits optical video or ultrasound images to an external display device 148 by wireless radio signal. While entering the external os 130 and proceeding through the cervical canal 132, the clinician may be guided by the imaging to navigate into and through the cervical canal.

As shown in FIG. 14B, the distal end of the catheterscope 138 is further advanced to exit the cervix canal 132 (at the internal os 134) and pass into the uterine cavity. An embryo catheter loaded with embryo(s) is inserted into the access port (not shown in this figure), passed through the working channel, and into the uterine cavity. The embryo(s) is released from the embryo catheter so that it can implant into the endometrial lining.

The foregoing description and examples have been set forth merely to illustrate the invention and are not intended to be limiting. Each of the disclosed embodiments of the invention may be considered individually or in combination with other embodiments or variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. An endoscopic instrument comprising:
a tubular body having an outer diameter of less than 2.5 mm and having a distal end;
at the distal end of the tubular body, a white light illuminator for providing illumination light;
within the tubular body, a working channel having an internal diameter of less 1.5 mm;
within the tubular body, a polymer optical fiber bundle for optical imaging;
a housing from which the tubular body extends out;
an imaging assembly in the housing, the imaging assembly comprising:
an camera module that receives optical images transmitted through the optical fiber bundle;
a short range radio transmitter that is coupled to the camera module and wirelessly transmits image data.

2. The endoscopic instrument of claim 1, wherein the white light illuminator is an LED element.

3. The endoscopic instrument of claim 1, wherein the white light illuminator is a phosphorescent element.

4. The endoscopic instrument of claim 1, wherein the working channel has an internal diameter of less 1.0 mm.

5. The endoscopic instrument of claim 1, wherein the tubular body has a length in the range of 15-28 cm.

6. The endoscopic instrument of claim 1, wherein outer diameter of tubular body is less than 2.0 mm.

7. The endoscopic instrument of claim 1, wherein the tubular body does not have a separate channel for suction, irrigation, or insufflation.

8. The endoscopic instrument of claim 1, wherein the working channel is the only hollow channel in the tubular body.

9. The endoscopic instrument of claim 1, wherein the imaging assembly further comprises a battery, and wherein the endoscopic instrument lacks a connector for an external power source.

10. The endoscopic instrument of claim 1, wherein:
the tubular body has a length L and extends out at a straight horizontal axis;
the tubular body is flexible, but with a flexibility range that is limited to a droop of less than 0.5×L from the straight horizontal axis at the distal end of the tubular body.

11. The endoscopic instrument of claim 1, wherein the tubular body is detachable from the housing and disposable.

12. An endoscopic instrument comprising:
a tubular body having an outer diameter of less than 2.5 mm and having a distal end;
at the distal end of the tubular body, an ultrasound transducer for ultrasound imaging;
within the tubular body, a working channel having an internal diameter of less 1.5 mm;
a housing from which the tubular body extends out;
an imaging assembly in the housing, the imaging assembly comprising:
a digital signal processor that receives ultrasound image signals from the ultrasound transducer;
a short range radio transmitter that is coupled to the digital signal processor and wirelessly transmits ultrasound image data.

13. The endoscopic instrument of claim 12, wherein outer diameter of tubular body is less than 2.0 mm.

14. The endoscopic instrument of claim 12, wherein the tubular body is detachable from the housing and disposable.

15. The endoscopic instrument of claim 12, wherein the tubular body has a length in the range of 15-28 cm.

16. A method of performing an embryo transfer procedure in assisted reproduction to deposit an embryo into a patient's uterine cavity, comprising:
having an endoscopic instrument of claim 1;
loading onto an embryo transfer catheter, an embryo;
inserting the tubular body of the endoscopic instrument into the patient's vagina;
advancing the tubular body through the external cervical os;
while viewing video images from the endoscopic instrument within the cervical canal, advancing the tubular body through the cervical canal;
exiting the cervical canal into the uterine cavity;
inserting the embryo transfer catheter into the working channel of the endoscopic instrument;
advancing the embryo transfer catheter through the working channel and into the uterine cavity;
releasing the embryo into the uterine cavity.

17. The method of claim 16, wherein the distal end of the endoscopic instrument is advanced through the external cervical os without prior dilation of the cervix.

18. The method of claim 16, further comprising detaching the tubular body and disposing of the tubular body after use.

19. The method of claim 16, further comprising disposing of the entire endoscopic instrument after use.

20. The method of claim 16, wherein the tubular body has a length in the range of 15-28 cm.
